(19) United States
(12) Patent Application Publication
(10) Pub. No.: US 2025/0241990 A1
(43) Pub. Date: Jul. 31, 2025

(54) THERAPEUTIC AGENT

(71) Applicant: SELECTIMMUNE PHARMA AB, Lund (SE)

(72) Inventors: Catharina Svanborg, Lund (SE); Ines Ambite, Lund (SE)

(21) Appl. No.: 18/833,811

(22) PCT Filed: Jan. 27, 2023
(86) PCT No.: PCT/EP2023/052076

§ 371 Date: Jul. 26, 2024

(30) Foreign Application Priority Data
Jan. 28, 2022 (GB) 2201139.9

(51) Int. Cl. A61K 38/20; A61K 31/16; A61K 31/381; A61K 31/65; A61K 38/16; A61K 38/17; A61P 13/02; A61P 13/12; A61P 31/04

(52) U.S. Cl. CPC A61K 38/2006; A61K 31/16; A61K 31/381; A61K 31/65; A61K 38/164; A61K 38/1793; A61P 13/02; A61P 13/12; A61P 31/04

(57) ABSTRACT

The present invention relates to methods and compositions for preventing and/or treating pyelonephritis and/or urosepsis.

### TECHNICAL FIELD

The present invention relates to methods and compositions for preventing and/or treating pyelonephritis and/or urosepsis.

### BACKGROUND OF THE INVENTION

Urinary tract infections (UTIs) are common and may be dangerous. The clinical presentation and severity varies depending on the site of infection and molecular basis of disease. In acute pyelonephritis (APN), bacteria ascend into the renal pelvis, where they cause an intense mucosal inflammatory response with progression into the renal parenchyma.

Symptoms include high fever, malaise, loin pain as well as poor feeding and irritability in infants. APN can lead to urosepsis.

In acute pyelonephritis, a pathogen-specific TLR4 response is activated by P fimbriated E. coli, through ceramide release and the successive phosphorylation of the TICAM-1 (TRIF) and TICAM-2 (TRAM) adaptors, CREB-1, c-FOS and c-JUN activates IRF- and API-dependent transcription. Additional involvement of MyD88, TIRAP and NF-κB depends on the virulence repertoire of the infecting strain. Genetic studies in the murine UTI model have identified IRF3-dependent gene expression and mCXCR2-dependent neutrophil activation as determinants of bacterial clearance and tissue homeostasis. Infected Irf3^{-/-} or mCxcrl^{-/-} mice develop severe APN and tissue damage after one week and relevance for human APN susceptibility has been demonstrated, through disease-associated IRF3 and CXCR1 polymorphisms in APN prone patients.

### SUMMARY OF THE INVENTION

The inventors have now surprisingly identified agents that can be used to treat or prevent pyelonephritis, including APN, and as a result to prevent resulting urosepsis. Those agents include inhibitors of IL-1 receptors, particularly IL-1B, and NIpD proteins. Without being bound by theory, based on their understanding of IL-1B processing, the inventors have also identified that MMP7 inhibitors and agents that moderate the expression of MMP7 may also be useful in the invention. It is particularly surprising that these agents, known for their anti-inflammatory activities can be used to treat infection.

The present invention provides a method for preventing or treating pyelonephritis and/or urosepsis comprising administering to a patient in need thereof, an effective amount of an agent selected from the group consisting of IL-1 inhibitors, MMP inhibitors and NIpD proteins.

The invention also provides an agent selected from the group consisting of IL-1 inhibitors, MMP inhibitors and NIpD proteins, for use in the treatment or prevention of pyelonephritis and/or urosepsis.

In certain embodiments, the method or agent is for the treatment or prevention, preferably the treatment, of pyelonephritis. In certain embodiments, the pyelonephritis is acute pyelonephritis. In certain embodiments, the pyelonephritis is chronic or long-term. In certain embodiments, the method or agent is for the treatment or prevention, preferably the prevention, of urosepsis, particularly urosepsis caused by pyelonephritis.

In certain embodiments, the agent may be provided in a pharmaceutical composition, comprising a pharmaceutically acceptable carrier.

In particular, the agent is an IL-1 inhibitor. In certain embodiments, it is an IL-1 inhibitor. In certain embodiments, it is an IL-1 receptor antagonist inhibitor. Many IL-1 inhibitors are known in the art. These include, for example, small molecules such as anthraquinones, described for example in U.S. Pat. No. 4,244,968 including diacerein, as well as proteins and peptides such as an interleukin-1 receptor antagonist (IL-1 RA), for example anakinra and rilonacept, or pharmaceutically acceptable salts thereof, or prodrugs thereof, and combinations of these. In particular, the agent is an IL-1B receptor antagonist, such as anakinra (U.S. Pat. No. 5,075,222).

Alternatively, the agent is an MMP inhibitor, and in particular an MMP7 inhibitor. A wide range of MMP inhibitors are known as described for example Durrant et al. Chem. Biol. Drug Des 20111; 78; 191-198, the content of which is incorporated herein by reference. Particular examples include batimastat, periostat (doxycycline hyclate), marimastat, or salts or prodrugs thereof, but in particular batimastat. It may also be an agent that reduces the expression of MMP, particularly MMP-7, such as a protein selected from ASC or NLRP-3, or an active fragment or variant thereof.

In certain embodiments, the NIpD protein is a bacterial protein, preferably a commensal bacteria or asymptomatic carrier. In certain embodiments, this is a commensal bacteria or asymptomatic carrier with respect to a human host. The bacteria may be asymptomatic bacteriuria (ABU). In certain embodiments, the bacteria strain is an E. coli strain, such as E. coli 83972.

In certain embodiments, the NIpD protein may comprise or consist of SEQ ID NO: 1 or a variant or active fragment thereof.

(SEQ ID NO: 1)
MSAGSPKFTV RRIAALSLVS LWLAGCSDTS NPPAPVSSVN
GNAPANTNSG MLITPPPKMG TTSTAQQPQI QPVQQPQIQA
TQQPQIQPMQ PVAQQPVQME NGRIVYNRQY GNIPKGSYSG
STYTVKKGDT LFYIAWITGN DFRDLAQRNN IQAPYALNVG
QTLQVGNASG TPITGGNAIT QADAAEQGVV IKPAQNSTVA
VASQPTITYS ESSGEQSANK MLPNNKPTAT TVTAPVTVPT
ASTTEPIVSS TSTSTPISTW RWPTEGKVIE TFGASEGGNK
GIDIAGSKGQ AIIATADGRV VYAGNALRGY GNLIIIKHND
DYLSAYAHND TMLVREQQEV KAGQKIATMG STGTSSTRLH
FEIRYKGKSV NPLRYLPQR

One particular fragment of SEQ ID NO: 1 is represented in bold (which is SEQ ID NO: 2).

In certain embodiments, the NIpD protein, or variant or active fragment thereof, is of low molecular weight, for instance less than 3 kDa in molecular weight. In other embodiments, the proteins can be larger, for example about 40 kDa.

As used herein, the expression ‘fragment’ refers to a peptide or protein which lacks one or more amino acids found in a full length protein but which still has the function of the full length protein.

The expression “variant” refers to proteins or polypeptides having a similar biological function but in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

“Conservative substitution” means the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt activity. Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the fundamental properties and activity of the basic polypeptide. For example, when determining whether a variant of the polypeptide falls within the scope of the invention, the skilled person will determine whether the variant retains the biological activity of the native protein and whether the variant has at least 60%, preferably at least 70%, more preferably at least 80%, yet more preferably 90%, 95%, 96%, 97%, 98%, 99% or 100% activity of the native protein.

Variants of the polypeptide may comprise or consist essentially of an amino acid sequence with at least 70% identity, for example at least 75%, 80%, 85%, 90%, 91%, 92%, 93%, 94%, 96%, 97%, 98% or 99% identity to a native polypeptide sequence. The level of sequence identity is suitably determined using the BLASTP computer program with the native polypeptide sequences as the base sequence. This means that native polypeptide sequences form the sequence against which the percentage identity is determined. The BLAST software is publicly available at http://blast.ncbi.nlm.nih.gov/Blast.cgi (accessible on 13 Oct. 2016).

The NIpD proteins may be isolated from bacteria. Where the NIpD protein is a variant or active fragment, it may be obtained by recombinant expression. Where the protein is obtained by recombinant expression, the protein sequence may comprise a sequence for use in purification, such as an N-terminal or C-terminal His tag. The use of purification tags is well-known in the art. In a preferred embodiment, the NIpD proteins, or variants or active fragments thereof, are synthetic. Typically, the NIpD protein, or variants or active fragments thereof, will be isolated or synthetic.

For administration to patients, the agent is suitably administered in the form of a pharmaceutical composition, which further comprise a pharmaceutically acceptable carrier. Such compositions are known in the art.

Suitable pharmaceutical compositions will be in either solid or liquid form. They may be adapted for administration by any convenient route, such as parenteral, oral or topical administration or for administration by inhalation or insufflation. The pharmaceutical acceptable carrier may include diluents or excipients which are physiologically tolerable and compatible with the active ingredient.

Parenteral compositions are prepared for injection, for example either subcutaneously or intravenously. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories or intranasal aerosols. For suppositories, traditional binders and excipients may include, for example, polyalkylene glycols or triglycerides; such suppositories may be formed from mixtures containing the active ingredient.

The amount of agent administered will vary depending upon factors such as the nature of the agent being used, the size and health of the patient, the nature of the condition being treated etc. in accordance with normal clinical practice. Typically, a dosage in the range of from \hat{ }g-50 mg/Kg for instance from 2-20 mg/Kg, such as from 5-15 mg/Kg would be expected to produce a suitable effect.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows the effect of NIpD and IL-1RA in infected Irf3^{-/-} mice, genetic model of acute pyelonephritis and urosepsis.

FIG. 1A shows the experimental protocol, 1B shows urine bacterial and neutrophil counts, 1C shows kidney gross pathology at sacrifice day and 1D shows kidney bacterial numbers at sacrifice day.

### DETAILED DESCRIPTION OF THE INVENTION

### Example 1

### Methods and Materials

Irf3^{-/-} mice were infected with E. coli CFT073 (50 μl of 2.109 cfu/ml) by intravesical instillation. Intraperitoneal treatment was administered daily, starting 6 hours after infection and for 7 days. Mice were treated with recombinant NIpD protein (105 μg in 100 μl) or IL-1RA (Anakinra, 1 mg/kg, 100 μl). Control mice received PBS. Urine sampling for bacterial and neutrophil counts were done on day 1, 3, 5 and 7 in one group of animals (those sacrificed on day 7), and on day 1, 3, 5, 7, 21 and 42 in others.

Animals were sacrificed under anesthesia; kidneys and bladders were asceptically removed and in the case of bladders, macroscopic pathology was documented by photography. Tissues were fixed with 4% paraformaldehyde or frozen for sectioning and RNA extraction. Viable counts in homogenized tissues (Stomacher 80, Seward Medical) were determined on TSA (37° C., overnight). Urine samples were collected prior to and at regular times after infection and quantitatively cultured. Neutrophils in uncentrifuged urine were counted, using a hemocytometer.

### Results

### Urine Bacterial and Neutrophil Counts.

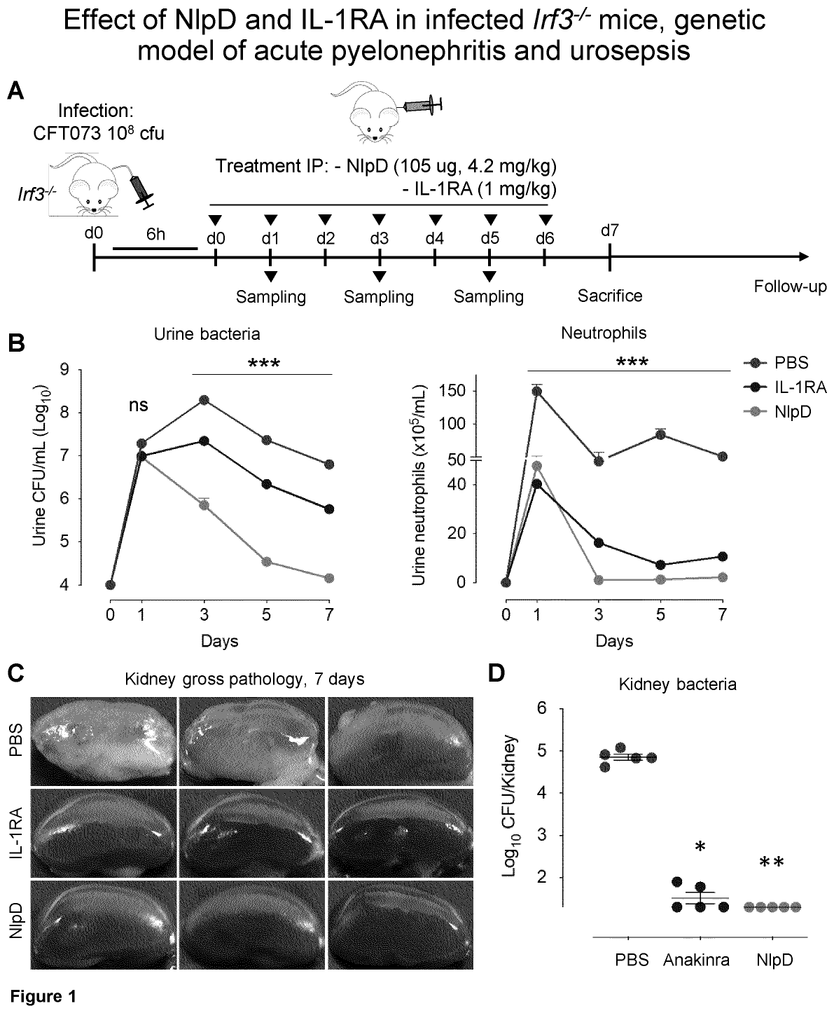

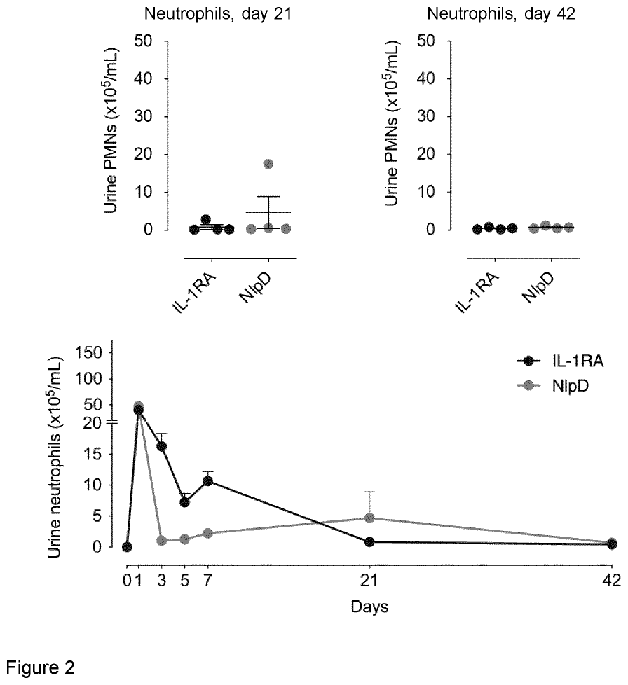

As shown in FIG. 1B, treatments increased bacterial clearance from the urinary tract and decreased urine neutrophil infiltration. Two-way ANOVA, and Sidak's multiple comparison test were used to assess the results.

### Kidney Gross Pathology.

Untreated Irf3^{-/-} controls developed severe kidney pathology with evidence of renal abscesses. Treatments protected the mice from kidney pathology.

### Kidney Bacterial Numbers (Kruskal-Wallis Tests).

Kidney pathology in untreated mice was combined with high bacterial counts. Treated mice had very low or no kidney bacterial growth.

### Urine Bacterial and Neutrophil Counts

As shown in FIG. 2, treatment provided long term protection, against infection and inflammation in mice treated with either IL-1RA or NIpD. Infected and treated mice remained disease free with no bacterial growth in urine at day 21 and 42, and had low urine neutrophil numbers.

1. An agent selected from the group consisting of IL-1 inhibitors, MMP inhibitors and NIpD proteins, for use in the treatment or prevention of pyelonephritis, particularly acute pyelonephritis and/or urosepsis.

2. A method for preventing or treating pyelonephritis, especially acute pyelonephritis, and/or urosepsis comprising administering to a patient in need thereof, an effective amount of an agent selected from the group consisting of IL-1 inhibitors, MMP inhibitors and NIpD proteins.

3. An agent according to claim 1, or a method according to claim 2, wherein the agent or method is for the treatment or prevention of acute pyelonephritis.

4. An agent according to claim 1, or a method according to claim 2, wherein the agent or method is for the prevention of urosepsis, caused by acute pyelonephritis.

5. An agent according to claim 1, 3 or 4, or a method according to claim 2, 3 or 4, wherein the agent an IL-1B inhibitor.

6. An agent according to claim 1, 3, 4 or 5, or a method according to claim 2, 3, 4 or 5, wherein the IL-1 inhibitor is an interleukin-1 receptor antagonist (IL-1 RA).

7. An agent or a method according to claim 6, wherein the IL-1RA is anakinra or rilonacept, or a pharmaceutically acceptable salt, or a prodrug thereof.

8. An agent according to claim 1, 3 or 4, or a method according to claim 2, 3 or 4, wherein the agent an MMP inhibitor.

9. An agent or a method according to claim 8, wherein the MMP inhibitor is an MMP7 inhibitor.

10. An agent or a method according to claim 8, wherein the MMP inhibitor is batimastat, periostat (doxycycline hyclate), marimastat, or a salt or prodrug thereof.

11. An agent according to claim 1, 3 or 4, or a method according to claim 2, 3 or 4, wherein the agent is an NIpD protein or fragment or variant thereof.

12. An agent or a method according to claim 11, wherein the NIpD protein is bacterial, particularly from a commensal bacteria or asymptomatic carrier.

13. An agent or a method according to claim 11, wherein the NIpD protein comprises or consists of an amino acid sequence selected from SEQ ID NO 1 or SEQ ID NO 2, or a variant or active fragment thereof.


SEQUENCE LISTING (2 sequences)

SEQ ID NO: 1 - protein, 379 aa, Escherichia coli
MSAGSPKFTVRRIAALSLVSLWLAGCSDTSNPPAPVSSVNGNAPANTNSGMLITPPPKMGTTSTAQQPQIQPVQQPQIQATQQPQIQPMQPVAQQPVQMENGRIVYNRQYGNIPKGSYSGSTYTVKKGDTLFYIAWITGNDFRDLAQRNNIQAPYALNVGQTLQVGNASGTPITGGNAITQADAAEQGVVIKPAQNSTVAVASQPTITYSESSGEQSANKMLPNNKPTATTVTAPVTVPTASTTEPIVSSTSTSTPISTWRWPTEGKVIETFGASEGGNKGIDIAGSKGQAIIATADGRVVYAGNALRGYGNLIIIKHNDDYLSAYAHNDTMLVREQQEVKAGQKIATMGSTGTSSTRLHFEIRYKGKSVNPLRYLPQR

SEQ ID NO: 2 - protein, 350 aa, Escherichia coli
SNPPAPVSSVNGNAPANTNSGMLITPPPKMGTTSTAQQPQIQPVQQPQIQATQQPQIQPMQPVAQQPVQMENGRIVYNRQYGNIPKGSYSGSTYTVKKGDTLFYIAWITGNDFRDLAQRNNIQAPYALNVGQTLQVGNASGTPITGGNAITQADAAEQGVVIKPAQNSTVAVASQPTITYSESSGEQSANKMLPNNKPTATTVTAPVTVPTASTTEPIVSSTSTSTPISTWRWPTEGKVIETFGASEGGNKGIDIAGSKGQAIIATADGRVVYAGNALRGYGNLIIIKHNDDYLSAYAHNDTMLVREQQEVKAGQKIATMGSTGTSSTRLHFEIRYKGKSVNPLRYLPQR